(19) United States
(12) Patent Application Publication
(10) Pub. No.: US 2023/0241354 A1
(43) Pub. Date: Aug. 3, 2023

(54) Skin Nicking Device for Catheter Placement System

(71) Applicant: Bard Access Systems, Inc., Salt Lake City, UT (US)

(72) Inventor: Glade H. Howell, Draper, UT (US)

(21) Appl. No.: 18/105,357

(22) Filed: Feb. 3, 2023

Related U.S. Application Data
(60) Provisional application No. 63/306,430, filed on Feb. 3, 2022.

(51) Int. Cl. A61M 25/06; A61M 25/01

(52) U.S. Cl. CPC A61M 25/065; A61M 25/0102; A61M 2207/00

(57) ABSTRACT

A catheter placement assembly includes a catheter and a skin nicking device having a blade configured to transition between a sheathed configuration and a deployed configuration. The skin nicking device includes a needle defining having a slot extending through the needle wall, where the blade is disposed within the slot. The is hingedly coupled to the needle wall defining a fulcrum. The skin nicking device includes a push rod within the needle lumen, having a distal end operatively engaged with the blade such displacement of the push rod between a proximal position and a distal position transitions the blade between the sheathed configuration and the deployed configuration, respectively.

### PRIORITY

This application claims the benefit of priority to U.S. Provisional Application No. 63/306,430, filed Feb. 3, 2022, which is incorporated by reference in its entirety into this application.

### BACKGROUND

Central venous catheter (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma or increased risk of infection. There is a relatively high potential for touch contamination due to the number of medical devices that need to be interchanged during the Seldinger technique. As such, advanced catheter placement systems have been developed to reduce the number of steps and medical devices involved in placing a catheter, such as a CVC, into a patient.

Some of these advanced catheter placement systems include accessing a vasculature with a needle and stabilizing the access site with a guidewire. Once the guidewire is placed, a scalpel may be used to cut or nick the skin and fascia at the insertion site to ease the insertion of the catheter. If the skin nick is not created properly, a skin bridge may form, impeding insertion of the catheter through the skin into the vessel. A skin nicking device may be used to create a repeatable depth of cut, reducing the likelihood of leaving skin bridges around the insertion site. Disclosed herein are advanced catheter placement systems and associated methods for nicking the skin at the insertion site to eliminate skin bridges impeding the insertion of the catheter into the vasculature.

### SUMMARY

Disclosed herein is a catheter placement device including a skin nicking device configured to enlarge an insertion site opening. According to some embodiments, the skin nicking device includes (i) a needle defining a needle lumen and a needle wall, where the needle includes a slot extending through the needle wall; a blade disposed within the needle lumen in alignment with the slot; and (iii) a push rod disposed within the needle lumen, where the push rod includes a distal portion configured to operatively engage the blade such that proximal displacement of the push rod causes radially outward displacement of the blade through the slot.

In some embodiments, the blade defines a sharp edge disposed opposite a dull edge, and the blade is oriented within the needle lumen such that the sharp edge is directed radially outward. In some embodiments, the sharp edge is disposed at an angle with respect to the dull edge such that a proximal width of the blade is greater than a distal width of the blade.

In some embodiments, the blade is transitionable between a sheathed configuration and a deployed configuration such that (i) in the sheathed configuration an entirety of the sharp edge is disposed radially inward of an outside surface of the needle and (ii) in the deployed configuration at least a portion of the sharp edge extends radially beyond the outside surface.

In some embodiments, the push rod is longitudinally positionable within the needle lumen between a proximal position and a distal position, and the distal portion of the push rod engages the blade such that distal displacement of the push rod away from the proximal position toward the distal position transitions the blade away from the sheathed configuration toward the deployed configuration.

In some embodiments, operative engagement of the distal portion with the blade includes sliding contact of the distal portion with the dull edge of the blade. In some embodiments, the distal portion includes an angled surface, and operative engagement of the distal portion with the blade includes sliding contact of the angled surface with the dull edge of the blade. In some embodiments, the distal portion includes a taper defining the angled surface. In some embodiments, the push rod includes channel along the distal portion, where the blade is disposed within the channel, and where a bottom surface of the channel defines the angled surface.

In some embodiments, a distal end of the blade is hingedly coupled with the needle wall defining a fulcrum such that the blade rotates between the sheathed configuration and the deployed configuration.

In some embodiments, the blade is biased toward the sheathed configuration such that proximal displacement of the push rod away from the distal position toward the proximal position allows the blade to self-rotate toward the sheathed configuration.

In some embodiments, the needle wall includes a push rod barrier protruding radially into the needle lumen, where the push rod barrier is configured to engage the push rod so as to limit distal displacement of the push rod beyond the distal position.

In some embodiments, the push rod includes a push rod lumen having an open proximal end and an open distal end, where the push rod lumen is configured for insertion of a guidewire therethrough. In some embodiments, the skin nicking device includes the guidewire disposed within the push rod lumen.

In some embodiments, the skin nicking device includes an actuator operatively coupled with the push rod, where the actuator is configured to displace the push rod between the proximal position and the distal position.

In some embodiments, the skin nicking device includes a locking mechanism configured to lock the push rod in at least one of the proximal position or the distal position.

Also disclosed herein is a catheter assembly that, according some embodiments, includes (i) a catheter including a catheter tube proximally coupled to a catheter hub having one or more extension legs proximally coupled therefrom, the catheter tube defining one or more lumens wherein each of the one or more lumens is in fluid communication with one of the extension legs and (ii) the catheter placement device according to any embodiment of described above. In some embodiments of the assembly, the needle of the catheter placement device is inserted into one of the one or more lumens of the catheter tube.

Also disclosed herein is a method of placing a catheter within a blood vessel of a patient that, according to some embodiments, includes (i) inserting a needle of a skin nicking device through a skin and into the blood vessel to define an insertion site, (ii) transitioning a blade of the skin nicking device from a sheathed configuration to a deployed configuration such that a sharp edge of the blade extends radially away from a outside surface of the needle, (iii) nicking the skin with the sharp edge to enlarge the insertion site, and (iv) inserting a catheter through the insertion site into the blood vessel.

In some embodiments of the method, the skin nicking device includes a push rod disposed within a needle lumen of the needle, where the push rod includes a distal portion configured to operatively engage the blade within the needle lumen such that distal displacement of the push rod causes radially outward displacement of the blade through a slot extending through a wall of the needle.

In some embodiments of the method, the blade is transitionable between a sheathed configuration and a deployed configuration such that in the sheathed configuration an entirety of the sharp edge is disposed radially inward of the outside surface of the needle, and in the deployed configuration at least a portion of the sharp edge extends radially beyond the outside surface.

In some embodiments of the method, the push rod is longitudinally positionable within the needle lumen between a proximal position and a distal position, where the distal portion engages the blade such that distal displacement of the push rod away from the proximal position toward the distal position transitions the blade away from the sheathed configuration toward the deployed configuration. In such embodiments, transitioning the blade of the skin nicking device from a sheathed configuration to the deployed configuration includes displacing the push rod from the proximal position to the distal position.

In some embodiments of the method, a distal end of the blade is hingedly coupled with the needle wall defining a fulcrum, and transitioning the blade between the sheathed configuration and the deployed configuration includes rotating the blade about the fulcrum.

In some embodiments, the method further includes displacing the push rod from the distal position toward the proximal position to transition the blade from the deployed configuration to the sheathed configuration.

In some embodiments of the method, the blade is biased toward the sheathed configuration such that proximal displacement of the push rod toward the proximal position allows the blade to self-rotate toward the sheathed configuration.

In some embodiments of the method, the push rod includes a push rod lumen extending between an open proximal end and an open distal end, where the push rod lumen is configured for insertion of a guidewire therethrough, and the method further comprises inserting the guidewire through the push rod lumen.

Also disclosed herein is a manufacturing method of a catheter placement device that, according to some embodiments, includes (i) forming a slot extending through a needle wall of a needle; (ii) placing a blade within the slot, where a sharp edge of the blade is directed radially outward with respect to the needle and the sharp edge is disposed inward of an outside surface of the needle; and (iii) inserting a push rod within a needle lumen of the needle such that an angled surface of the push rod engages the blade.

In some embodiments, manufacturing method further includes hingedly coupling the blade to the needle wall such that the blade is pivotable through the slot.

In some embodiments, manufacturing method further includes forming a protrusion on an inside surface of the needle lumen to define a push rod barrier configured to limit distal displacement of the push rod within the needle lumen.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a catheter placement system in an unfolded configuration, in accordance with embodiments disclosed herein;

FIG. 1B shows a plan view of a catheter placement system in a folded configuration ready for use, in accordance with embodiments disclosed herein;

FIG. 1C shows a perspective view of a catheter placement system in a folded configuration, in accordance with embodiments disclosed herein;

FIG. 2 shows a side view of a catheter of a catheter placement system in an unfolded configuration, in accordance with embodiments disclosed herein;

FIG. 3A shows close up detail of a distal portion of the catheter of FIG. 2, in accordance with embodiments disclosed herein;

FIGS. 3B-3C show cross-section views of the catheter of FIG. 3A, in accordance with embodiments disclosed herein;

FIG. 4 shows a longitudinal cross-section view of a distal portion of a catheter placement system, in accordance with embodiments disclosed herein.

FIGS. 5A-5E show an exemplary method of use for a catheter placement system, in accordance with embodiments disclosed herein;

FIG. 6A illustrates a cross sectional view of a skin nicking device, in accordance with some embodiments;

FIG. 6B is a side view of a distal portion of the push rod of FIG. 6A illustrating a second embodiment of the angled surface, in accordance with some embodiments;

FIG. 6C is a side view of a distal portion of the push rod of FIG. 6A illustrating a third embodiment of the angled surface, in accordance with some embodiments;

FIGS. 7A-7B illustrate cross sectional views of the skin nicking device in a concealed configuration and a deployed configuration, in accordance with some embodiments;

FIG. 8 illustrates a flow chart of an exemplary method of placing a catheter using the catheter insertion system, in accordance with some embodiments; and

FIG. 9 illustrates a flow chart of an exemplary manufacturing method of the skin nicking device of FIG. 6A, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

Unless defined otherwise, all technical and scientific terms used herein have the same meaning as commonly understood by those of ordinary skill in the art.

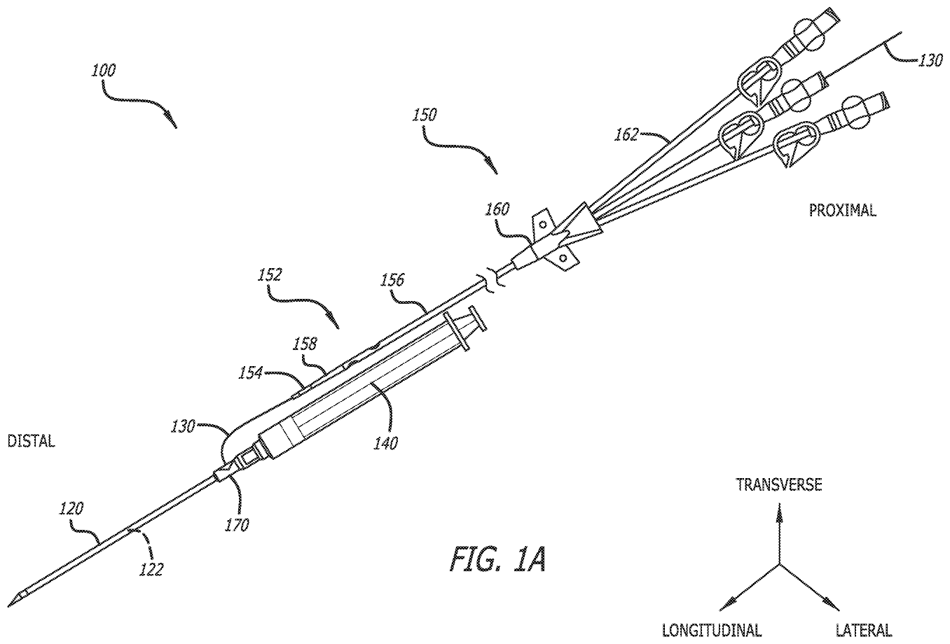

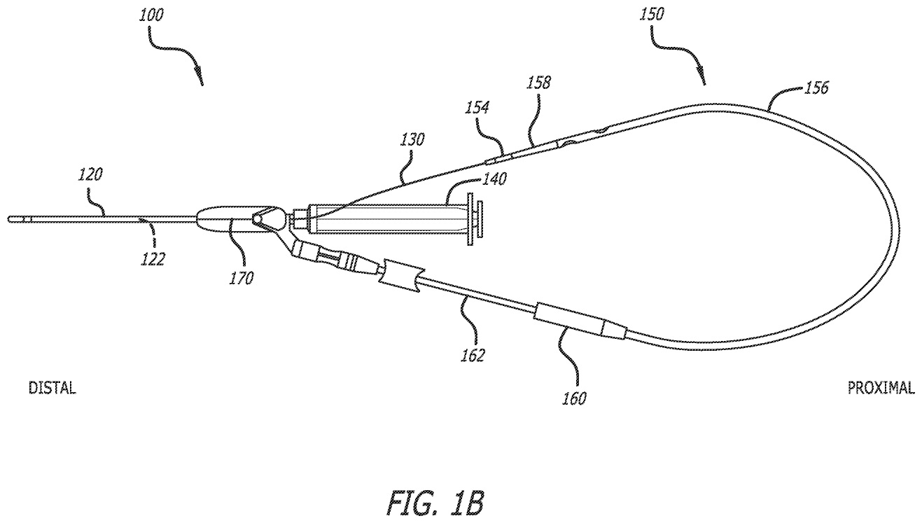

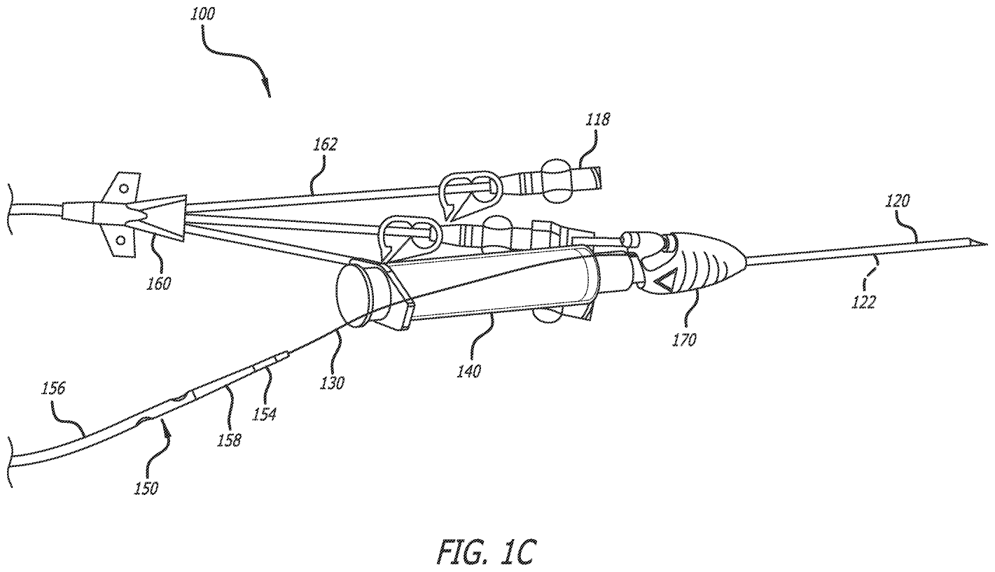

FIGS. 1A-1C show an exemplary advanced catheter placement system (“system”) 100, generally including a needle 120, a guidewire 130, a syringe system 140, a catheter 150, and a needle housing (“housing”) 170. FIG. 1A shows the system 100 in an unfolded configuration for ease of illustration. FIG. 1B shows a plan view of the system 100 in a folded configuration ready for use. FIG. 1C shows a perspective view the system 100 in a folded configuration. In an embodiment the catheter placement system 100 can be a Rapidly Insertable Central Catheter (RICC) placement system 100 configured to place a RICC 150. However, it will be appreciated that other catheter placement systems configured to place other types of catheters are also contemplated. Exemplary catheters 150 can also include peripheral intravenous (PIV) catheters, peripherally inserted central catheter (PICC), central venous catheters (CVC), midline catheters, dialysis catheters, single lumen catheters, multi-lumen catheters, or the like.

In an embodiment, the catheter 150 can generally include a catheter body 152 supported at a proximal end by a catheter hub (“hub”) 160. The hub 160 can include one or more extension legs 162 extending proximally therefrom. Each extension leg of the one or more extension legs 162 can be in fluid communication with a lumen of the catheter body 152. The catheter body 152 can include a first section 154 disposed distally, a second section 156 disposed proximally, and a transition section 158 disposed therebetween. The first section 154 can define a single lumen and have a first outer diameter, the second section 156 can define two or more lumen and can have a second diameter larger than the first diameter. The transition section 158 disposed between the first section 154 and the second section 156 can define a tapered shape extending from the first diameter of the first section to the second diameter of the second section. A guidewire 130 can extend through a lumen of the catheter 150 from a proximal end of an extension leg 162, to a distal tip of the first section 154.

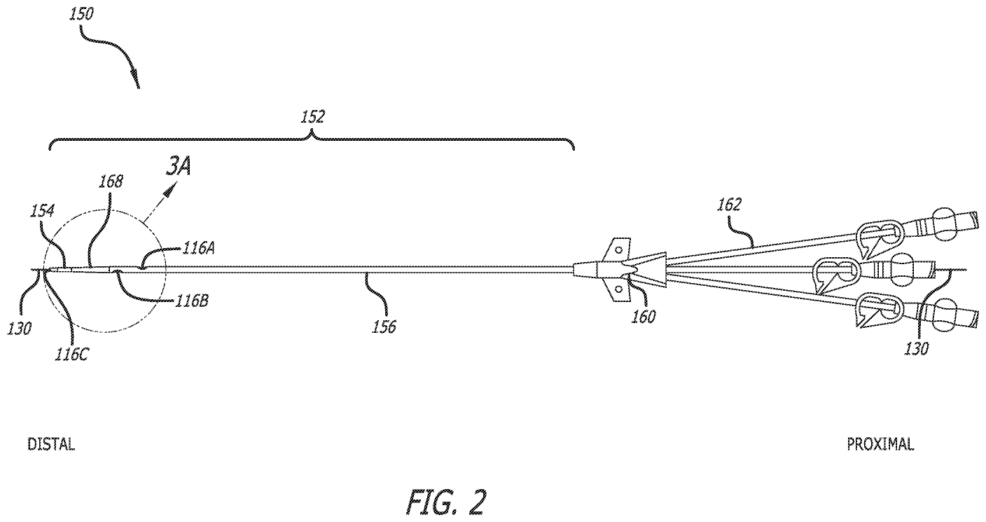

FIG. 2 shows further details of an exemplary catheter 150 of the system 100. As described herein, different sections of the catheter 150 are required to perform different functions and as such are required to display different mechanical properties. For example, the first section 154 and the transition section 158 can provide a more rigid mechanical properties or harder durometer material relative to the second section 156. As such, the first section 154 and transition section 158 can withstand greater axial forces without kinking or collapsing, as theses sections are urged distally, forming and dilating the insertion site. The second section 156 can be formed of a softer durometer, or a more compliant material to facilitate negotiating the second section 156 through tortuous vascular pathways.

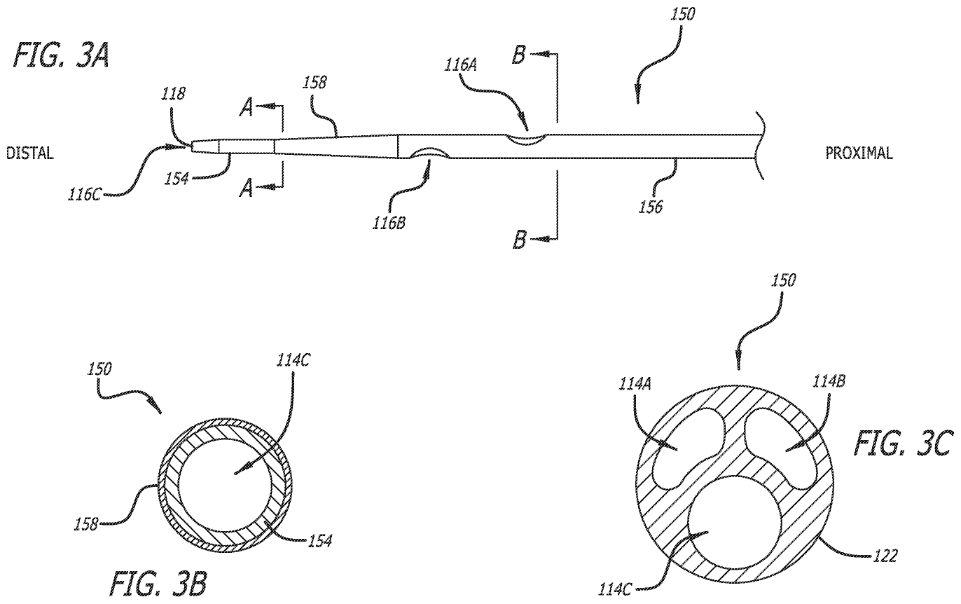

FIGS. 3A-3C show further details of a distal portion of the catheter 150, including the first section 154, the second section 156, and the transition section 158. In an embodiment, the second section 156 can include a proximal lumen 114A terminating at a proximal lumen aperture 116A, and a medial lumen 114B terminating at a medial lumen aperture 116B. Each of the proximal lumen aperture 116A and the medial lumen aperture 116B can extend through a side wall of the second section 156. Each of the proximal lumen aperture 116A and the medial lumen aperture 116B can be disposed proximally of the transition section 158. The proximal lumen aperture 116A can be disposed proximally of the medial lumen aperture 116B.

FIG. 3B shows a cross section view of the catheter body 152 at point “A” of FIG. 3A. As shown, the first section 154 can define a single lumen and a relatively smaller outer diameter. In an embodiment, a proximal portion of the first section 154 can be received within a distal portion of the transition section 158. A distal lumen 114C of the catheter 150 can extend to a distal tip 118 of the catheter 150 and can communicate with a distal lumen aperture 116C. FIG. 3C shows a cross section view of the second section 156 at point “B” of FIG. 3A, showing the proximal lumen 114A, medial lumen 114B and distal lumen 114C.

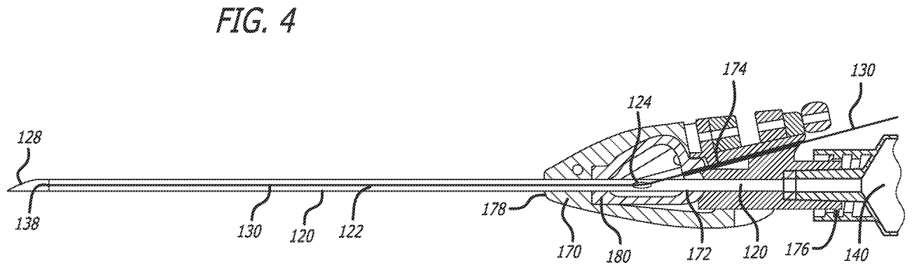

FIG. 4 shows a longitudinal cross-section view of a distal portion of a catheter placement system 100 including the needle 120, guidewire 130, a distal portion of the syringe system 140, and needle housing (“housing”) 170 including a needle splitter system 180, as described in more detail herein. In an embodiment, a proximal end of the needle 120 can be supported by a needle hub which can be coupled to, and supported by, a distal end of the syringe system 140. The syringe system 140 can be in fluid communication with needle lumen 122. The syringe system 140 can be configured to form a vacuum therein and draw a fluid flow proximally through the needle lumen 122. In an embodiment, the needle 120 can include a guidewire aperture 124 disposed in a wall of the needle 120 and communicating with a needle lumen 122. A distal portion of the guidewire 130 can extend through the guidewire aperture 124 and into the needle lumen 122. In an embodiment, a distal tip 138 of the guidewire 130 can be disposed proximate a distal tip 128 of the needle 120. As such, once the needle 120 accesses the vasculature, the distal tip 138 of the guidewire 130 can be positioned within the vasculature, expediting the placement of the catheter 150.

In an embodiment, the catheter placement system 100 can include a housing 170. The housing 170 can include a housing lumen 172 extending between a proximal end 176 and a distal end 178 of the housing 170. The housing 170 can further include a guidewire lumen 174 communicating with the housing lumen 172 and extending at an angle therefrom. A portion of the needle 120 can slidably engage the housing lumen 172. Further, the proximal end 176 of the housing can releasably engage one or both of a needle hub and a distal portion of the syringe system 140. When the housing 170 is engaged with the syringe system 140 the guidewire aperture 124 of the needle 120 can align with the guidewire lumen 174 of the housing 170. As such, the guidewire 130 can extend through the guidewire lumen 174 of the housing 170, through the guidewire aperture 124 of the needle 120 and into the needle lumen 122.

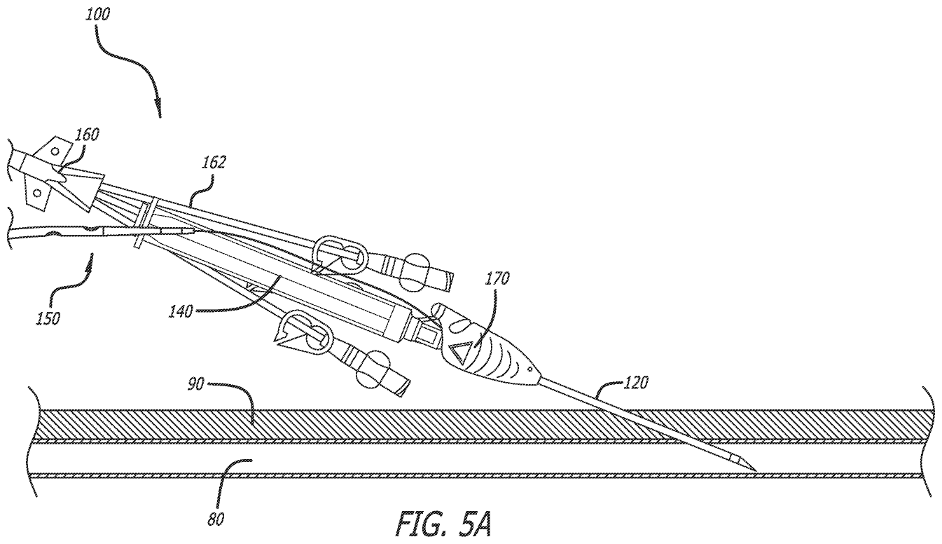

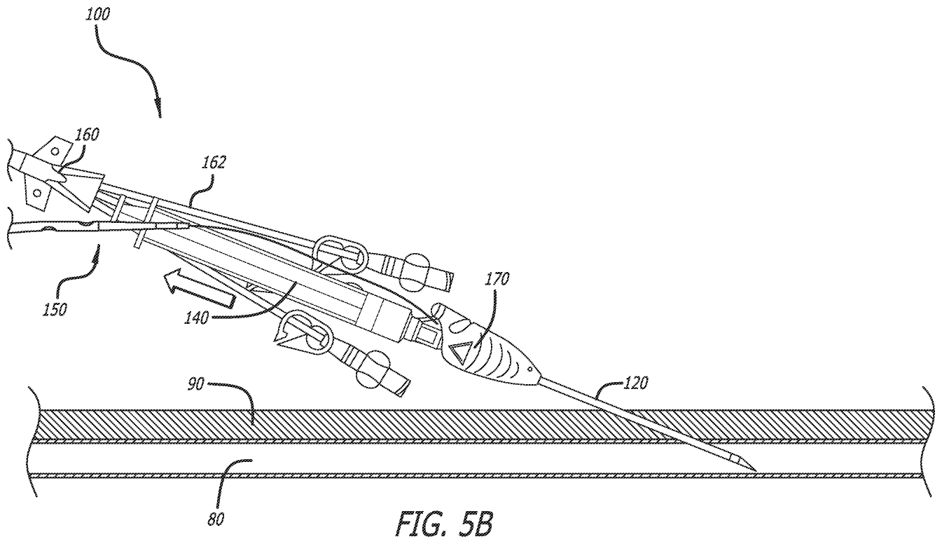

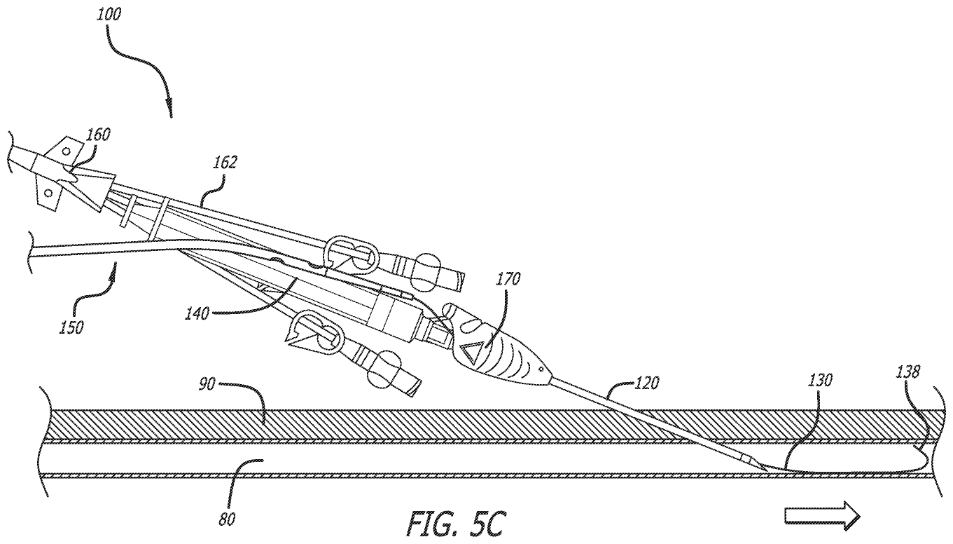

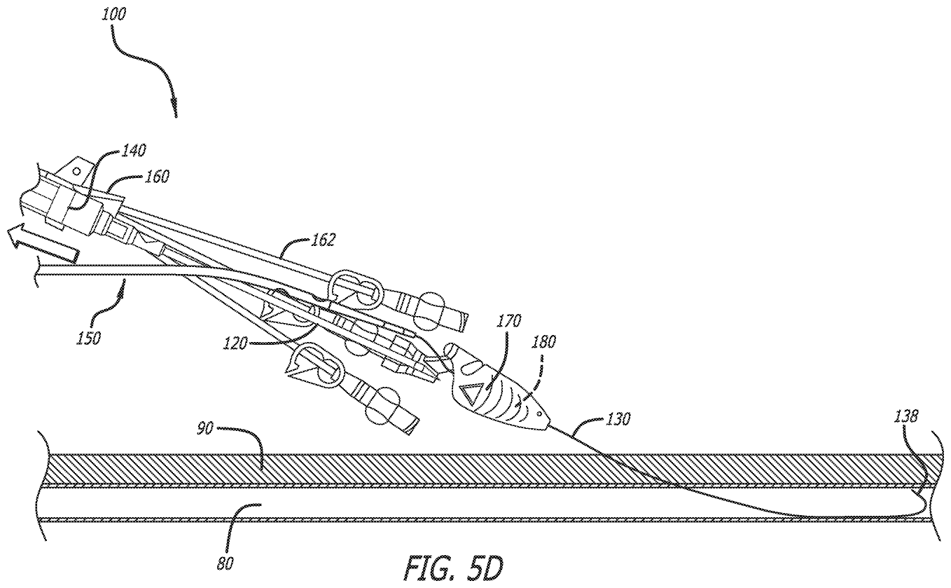

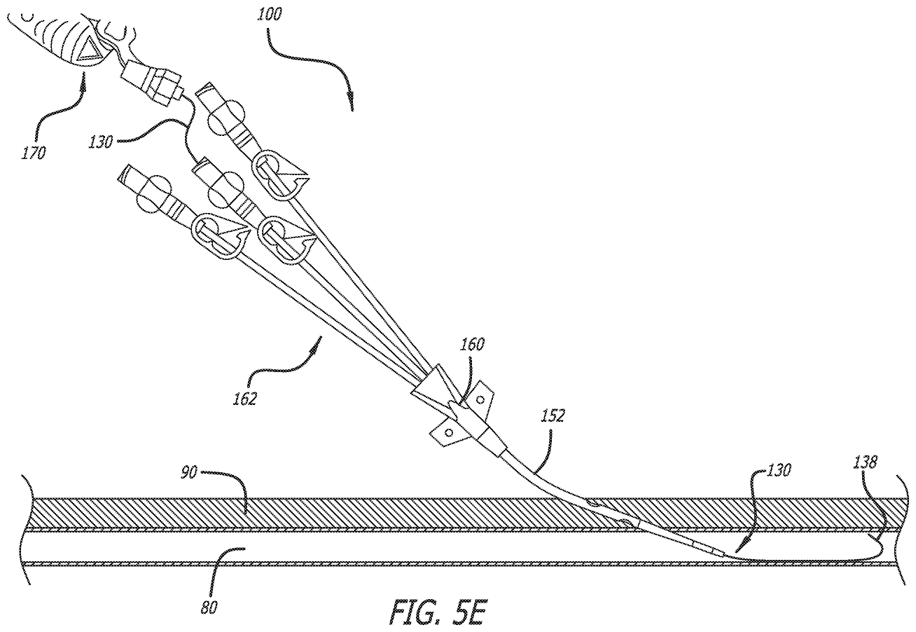

FIGS. 5A-5E show an exemplary method of placing a catheter 150 using the catheter placement system 100. As shown in FIG. 5A, the needle 120 can penetrate surface tissues 90 of the patient and access a vasculature 80, forming an insertion site. As shown in FIG. 5B, a syringe system 140, or similar device can form a vacuum and draw a fluid flow proximally through a needle lumen 122. A user can observe a color or pulsatile flow and confirm correct vascular access. Where incorrect vascular access is confirmed, the needle 120 can be withdrawn and the insertion site can be closed. As shown in FIG. 5C, once correct vascular access has been confirmed, the guidewire 130 can then be advanced through the needle lumen 122 and into the vasculature 80 to maintain patency of the insertion site.

As shown in FIG. 5D, the needle 120 and syringe system 140 assembly can be withdrawn proximally to disengage the needle 120 from the guidewire 130 while leaving a distal portion of the guidewire 130 in place within the vasculature 80. As described in more detail herein, the housing 170 can include a splitter system 180 configured to split the needle 120 longitudinally, as the needle 120 is withdrawn proximally. A portion of the guidewire 130 can pass between the two halves of the needle 120 to allow the needle 120 to disengage the guidewire 130.

As shown in FIG. 5E, with the needle 120 and syringe system 140 assembly disengaged from the guidewire 130, the catheter 150 can then be advanced over the guidewire 130 and into the vasculature. The first section 154 of the catheter 150, having only a single lumen and defining a relatively smaller outer diameter, can enter the vasculature 80 over the guidewire 130, anchoring the insertion site. The transition section 158 can then be urged distally, dilating the insertion site to allow the relatively larger diameter second section 156, defining two or more lumen, to enter the vasculature 80. Once the catheter 150 has been placed, the guidewire 130 can be withdrawn proximally.

Further details and embodiments of such catheter placement systems 100 can be found, for example, in U.S. Pat. No. 10,376,675, US 2019/0255294, US 2021/0069471, US 2021/0085927, US 2021/0113809, US 2021/0113810, US 2021/0121661, US 2021/0121667, US 2021/0228843, US 2021/0322729, US 2021/0330941, US 2021/0330942, US 2021/0361915, US 2021/0402153, US 2021/0402149, US 2022/0001138, U.S. patent application Ser. No. 17/390,682 filed Jul. 30, 2021, and U.S. Provisional Patent Application No. 63/229,862 filed Aug. 5, 2021, each of which is incorporated by reference in its entirety into this application.

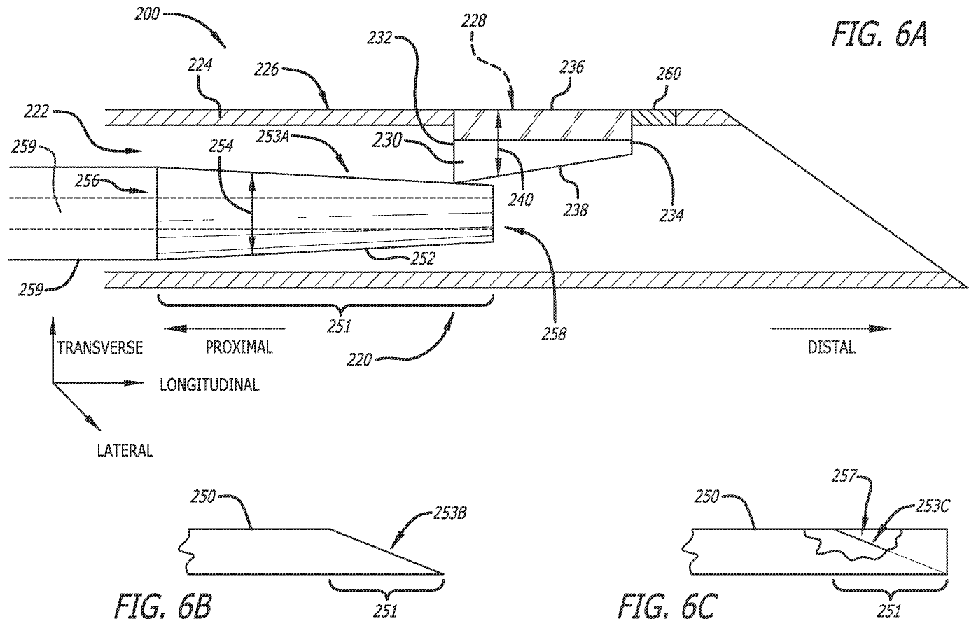

FIG. 6A illustrates a cross sectional view of a skin nicking device 200, in accordance with some embodiments which may generally constitute a catheter placement device. The skin nicking device 200 may be used in concert with the catheter placement system 100. In some embodiments, the skin nicking device 200 may be integrated into the catheter placement system 100. In some embodiments, the skin nicking device 200 may be pre-attached or pre-integrated into the catheter placement system 100. Similarly, the skin nicking device 200 may be coupled with a catheter, such as the catheter 150, to define a catheter assembly. In some embodiments of the catheter assembly, the skin nicking device 200 may be inserted within a lumen of the catheter.

The skin nicking device 200 includes a needle 220 having a needle lumen 222 therethrough. The needle 220 defines a needle wall 224 that includes a slot (or opening) 228 extending through the needle wall 224 between the needle lumen 222 and an outside surface 226 of the needle 220. The skin nicking device 200 further includes a blade 230 and a push rod 250, where each are at least partially disposed within the needle lumen 222 and where the blade 230 is in physical contact with the push rod 250. In some embodiments, the skin nicking device 200 may include more than one blade 230 in physical contact with the push rod 250. In some embodiments, wherein the skin nicking device 200 includes more than one blade 230, the needle 220 may correspondingly include more than one slot 228, e.g., a slot 228 for each blade 230. In some embodiments, each set of the blade 230 and the slot 228 may be laterally or transversely offset from the other set(s) of blade 230 and slot 228.

The push rod 250 may be configured to transition the blade 230 from a sheathed configuration to a deployed configuration, as will be described in more detail herein. The blade 230 includes a sharp edge 236 and a dull edge 238 opposite the shape edge 236. The blade 230 may be aligned with the slot 228 so that portions of the blade 230 or the entire blade 230 may be displaced through the slot 228. The sharp edge 236 is directed radially outward with respect to the needle 220 and the dull edge 238 is directed radially inward. The blade 230 defines a proximal end 232 and a distal end 234.

In some embodiments, the distal end 234 of the blade 230 is hingedly coupled to the needle wall 224 defining a fulcrum 260 such that the blade 230 is rotatable or pivotable about the fulcrum 260 through the slot 228. In some embodiments, the fulcrum 260 may be configured to allow the proximal end 232 including portions of the sharp edge 236 of the blade 230 to rotate out of the needle lumen 222 through the slot 228 such the sharp edge 236 or portion thereof extends radially outward of the outside surface 226. In some embodiments, the blade 230 may be tapered (i.e., the sharp edge 236 is disposed at an angle with respect to the dull edge 238 such that a blade width 240 at the proximal end 232 is greater than the blade width 240 at the distal end 234.

The push rod 250 includes a push rod body 252 including a distal portion 251. In the illustrated embodiment, the distal portion 251 defines a conical taper such that a push rod diameter 254 at the proximal end of the distal portion 251 is greater than the push rod diameter 254 and the distal end 258 of the distal portion 251. In the illustrated embodiment, the conical taper defines an angled surface 253A configured to engage the blade 230 (e.g., slidably contact the blade 230). In some embodiments, the distal portion 251 may have a consistent rate of taper from the proximal end to the distal end 258 of the distal portion 251. In some embodiments, the consistent rate of taper may be configured to allow the blade 230 to smoothly slide along the angled surface 253A to thereby smoothly transition the blade 230 between the sheathed configuration and the deployed configuration. The push rod 250 may be urged (or displaced) distally or proximally along the needle lumen 222. In some embodiments, the push rod 250 may be manually moved through the needle lumen 222. In other embodiments, the push rod 250 may be coupled to an actuator (not shown), e.g., a spring, a button, or the like, that displaces the push rod 250 along the needle lumen 222 or enables the user to displace the push rod 250 along the needle lumen 222. In some embodiments, the push rod 250 may be rigid from the proximal end (not shown) to the distal end 258 of the distal portion 251/push rod 250. In other embodiments, the push rod 250 may include one or more flexible portions and/or one or more rigid portions. In some embodiments, the angled surface 253A may slide along the dull edge 238 of the blade 230, or vice versa, to displace the sharp edge 236 of the blade 230 radially outward through the slot 228.

In some embodiments, the push rod 250 may include a push rod lumen 259 extending along the entire length of the push rod 250 or portion thereof, where the push rod lumen 259 defines an open proximal end and an open distal end. The push rod lumen 259 is configured to allow a guidewire (e.g., the guidewire 130) to be threaded therethrough. In some embodiments, the skin nicking device 200 include the guidewire inserted within the push rod lumen 259. The push rod 250 may extend through a portion or an entirety of the needle lumen 222. In some embodiments, the push rod 250 may be removable from the needle lumen 222. For example, the push rod 250 may be extractable from a proximal end of the needle lumen 222. In some embodiments, the angled surface 253A may include one or more notches (not shown) along the distal portion 251 or the angled surface 253A, wherein each notch corresponds to an extended distance of the blade 230 beyond the outside surface 226. In some embodiments, the proximal end 232 of the blade 230 may operatively engage each notch defining detent as the push rod 250 is distally displaced, where in some embodiments, the detent provides tactile feedback. In use, the one or more notches may enable the user to variably define a depth of a nick/cut of the skin.

FIG. 6B is side view of the distal portion 251 of the push rod 250 showing a second embodiment of the angled surface 253B. The angled surface 253B is formed of an angular cut across the push rod 250 so as to define a flat angled surface 253B extending along the distal portion 251.

FIG. 6C is side view of the distal portion 251 of the push rod 250 with a front half portion of the push rod 250 cut away showing a third embodiment of the angled surface 253C. In accordance with this embodiment, the push rod 250 includes a channel (or slot) 257 extending along the distal portion 251, where the bottom of the channel 257 defines the angled surface 253C. The channel 257 is configured to enable the blade 230 to slidably fit therein such that the dull edge 238 of the blade 230 may slide along the angled surface 253C.

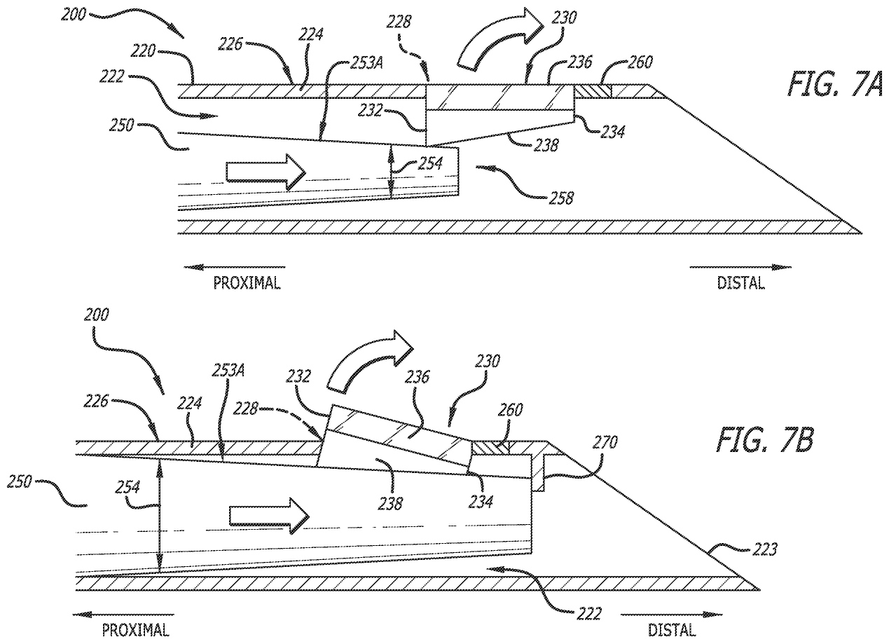

FIGS. 7A-7B illustrate cross sectional views of the skin nicking device 200 showing the blade 230 transitioning from the sheathed configuration to the deployed configuration, in accordance with some embodiments. FIG. 7A shows (i) the blade 230 in the sheathed configuration and (ii) the push rod 250 in a corresponding proximal position. As illustrated in FIG. 7A, the angled surface 253A may engage/contact the proximal end 232 of dull edge 238 of the blade 230. In the sheathed configuration, the entirety of the blade 230 is disposed beneath the outside surface 226. In some embodiments, the blade 230 may be biased towards the sheathed configuration, such as via the coupling between the blade 230 and the needle wall 224. In an embodiment, the skin nicking device 200 may include a locking mechanism (not shown), such as a clip or a detent, for example, to latch the push rod 250 in the proximal location.

FIG. 7B shows the blade 230 in the deployed configuration with the push rod 250 disposed in a corresponding distal position. In use, the user may displace the push rod 250 distally from the proximal position to the distal position to transition the blade 230 from the sheathed configuration to the deployed configuration. As illustrated in FIG. 7B, the dull edge 238 of the blade 230 slides along the angled surface 253A to displace blade 230 radially outward through the slot 228. Furthermore, with the distal end 234 of the blade 230 coupled to the needle wall 226 defining the fulcrum 260 so that the blade 230 is rotatably displaced through the slot 228 as the push rod 250 is displaced distally toward the distal position. Once the blade 230 is in the deployed configuration, the needle 220 may be used to nick/cut the skin or other body tissues 90 (FIG. 5A). In some embodiments, the locking mechanism may latch the push rod 250 in the distal location, thereby latching the blade 230 in the deployed configuration.

In some embodiments, longitudinal displacement of the push rod 250 may be restricted or limited within the needle lumen 222. For example, in an embodiment, a push rod barrier 270 may be suspended/protruded inward from the needle wall 224 within the needle lumen 220. The push rod barrier 270 may be configured to limit distal displacement of the push rod 250 within the needle lumen 222 beyond the distal position. In some embodiments, the push rod 250 may be pre-disposed at the proximal position. In an embodiment, the push rod barrier 270 may be configured to latch or temporality attach to the push rod 250 when the push rod 250 physically contacts the push rod barrier 270. In an embodiment, the push rod barrier 270 may latch to the push rod 250 in a snap fit, an interference fit, or the like. In an embodiment, the push rod barrier 270 may have magnetic elements and the push rod 250 may have ferrous elements, allowing the push rod barrier 270 to be magnetically latched to the push rod 250 when the push rod 250 is in the distal position.

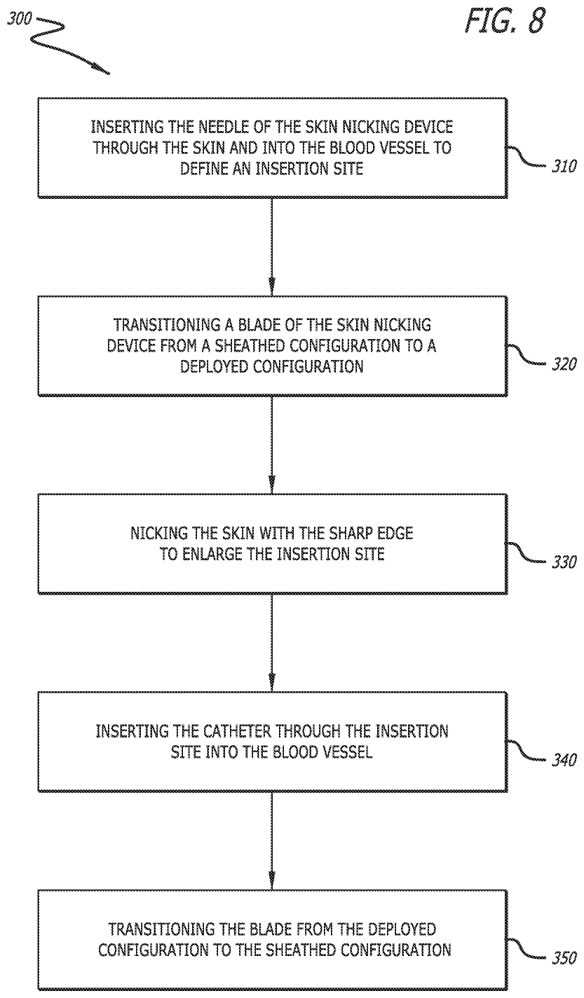

FIG. 8 illustrates a flow chart of an exemplary method 300 of placing a catheter within a blood vessel of a patient that includes all or any subset of the following steps, actions, or processes. The method 300 may include inserting the needle of the skin nicking device through the skin and into the blood vessel to define an insertion site (block 310).

The method 300 may further include transitioning the blade of the skin nicking device from a sheathed configuration to the deployed configuration (block 320) such that the sharp edge of the blade extends radially away from the outside surface of the needle. In some embodiments of the method 300, the blade is transitionable between the sheathed configuration and the deployed configuration such that in the sheathed configuration an entirety of the sharp edge is disposed radially inward of the outside surface of the needle, and in the deployed configuration at least a portion of the sharp edge extends radially beyond the outside surface. In some embodiments of the method 300, the skin nicking device includes a push rod disposed within the needle lumen of the needle, where the push rod includes a distal portion configured to operatively engage the blade within the needle lumen such that distal displacement of the push rod causes radially outward displacement of the blade through the slot of the needle wall. In some embodiments of the method 300, the push rod is longitudinally positionable within the needle lumen between the proximal position and the distal position, where the distal portion engages the blade such that distal displacement of the push rod away from the proximal position toward the distal position transitions the blade away from the sheathed configuration toward the deployed configuration. In such embodiments, transitioning the blade from the sheathed configuration to the deployed configuration includes displacing the push rod from the proximal position to the distal position. In some embodiments of the method 300, the distal end of the blade is hingedly coupled with the needle wall defining the fulcrum, and transitioning the blade between the sheathed configuration and the deployed configuration includes rotating/pivoting the blade about the fulcrum.

The method 300 may further include nicking the skin with the sharp edge to enlarge the insertion site (block 330), and further include inserting the catheter through the insertion site into the blood vessel (block 340).

The method 300 may further include transitioning the blade from the deployed configuration to the sheathed configuration (block 350) which may include displacing the push rod from the distal position toward the proximal position to transition the blade from the deployed configuration to the sheathed configuration. In some embodiments of the method 300, the blade is biased toward the sheathed configuration such that proximal displacement of the push rod toward the proximal position allows the blade to self-rotate toward the sheathed configuration.

In some embodiments of the method 300, the push rod includes a push rod lumen extending between an open proximal end and an open distal end, where the push rod lumen is configured for insertion of a guidewire therethrough, and the method 300 may further include inserting the guidewire through the push rod lumen. In such embodiments, the method 300 further include extracting the guidewire from the push rod lumen, such displacing the skin nicking device proximally along the guidewire until the skin nicking device is separated from the guidewire.

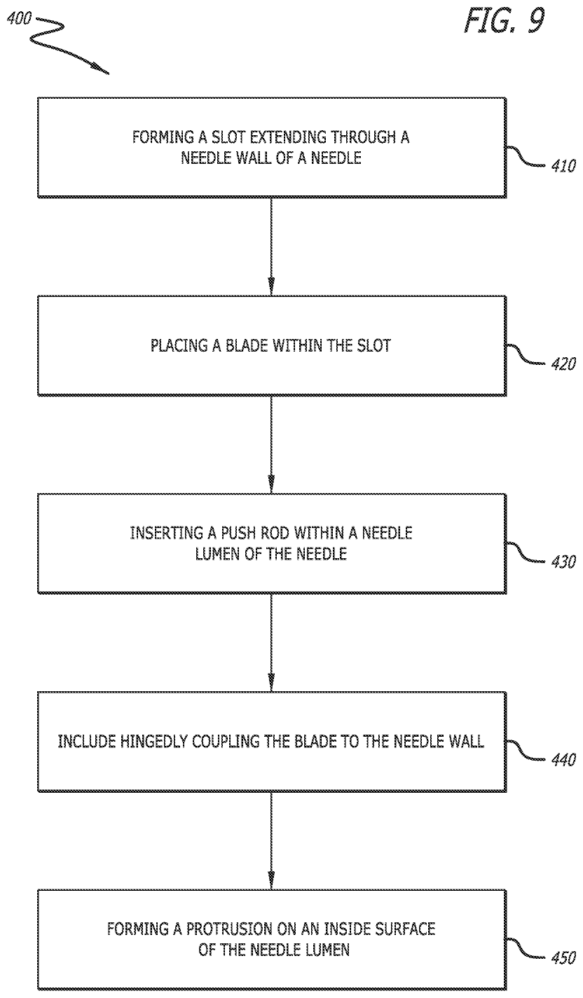

FIG. 9 illustrates a flow chart of an exemplary manufacturing method of the skin nicking device. The manufacturing method 400 may include forming a slot extending through a needle wall of a needle (block 410). The manufacturing method 400 may further include placing a blade within the slot (block 420), where the sharp edge of the blade is directed radially outward with respect to the needle and the sharp edge is disposed inward of an outside surface of the needle. The manufacturing method 400 may further include inserting a push rod within a needle lumen of the needle (block 430) such that an angled surface of the push rod engages the blade. The manufacturing method 400 may further include hingedly coupling the blade to the needle wall (block 440) such that the blade is pivotable through the slot. The manufacturing method 400 may further include forming a protrusion on an inside surface of the needle lumen (block 450) to define a push rod barrier configured to limit distal displacement of the push rod within the needle lumen.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A catheter placement device, comprising:
a skin nicking device configured to enlarge an insertion site opening, comprising:
a needle defining a needle lumen and a needle wall, the needle including a slot extending through the needle wall;
a blade disposed within the needle lumen in alignment with the slot; and
a push rod disposed within the needle lumen, the push rod including a distal portion configured to operatively engage the blade such that proximal displacement of the push rod causes radially outward displacement of the blade through the slot.

2. The device according to claim 1, wherein:
the blade defines a sharp edge disposed opposite a dull edge, and
the blade is oriented within the needle lumen such that the sharp edge is directed radially outward.

3. The device according to claim 2, wherein the sharp edge is disposed at an angle with respect to the dull edge such that a proximal width of the blade is greater than a distal width of the blade.

4. The device according to claim 2, wherein the blade is transitionable between a sheathed configuration and a deployed configuration such that:
in the sheathed configuration an entirety of the sharp edge is disposed radially inward of an outside surface of the needle, and
in the deployed configuration at least a portion of the sharp edge extends radially beyond the outside surface.

5. The device according to claim 4, wherein:
the push rod is longitudinally positionable within the needle lumen between a proximal position and a distal position, and
the distal portion engages the blade such that distal displacement of the push rod away from the proximal position toward the distal position transitions the blade away from the sheathed configuration toward the deployed configuration.

6. The device according to claim 2, wherein operative engagement of the distal portion with the blade includes sliding contact of the distal portion with the dull edge of the blade.

7. The device according to claim 6, wherein:
the distal portion includes an angled surface, and
operative engagement of the distal portion with the blade includes sliding contact of the angled surface with the dull edge of the blade.

8. The device according to claim 7, wherein the distal portion includes a taper defining the angled surface.

9. The device according to claim 7, wherein:
the push rod includes channel along the distal portion,
the blade is disposed within the channel, and
a bottom surface of the channel defines the angled surface.

10. The device according to claim 4, wherein a distal end of the blade is hingedly coupled with the needle wall defining a fulcrum such that the blade rotates between the sheathed configuration and the deployed configuration.

11. The device according to claim 10, wherein the blade is biased toward the sheathed configuration such that proximal displacement of the push rod away from the distal position toward the proximal position allows the blade to self-rotate toward the sheathed configuration.

12. The device according to claim 5, wherein the needle wall includes a push rod barrier protruding radially into the needle lumen, the push rod barrier configured to engage the push rod so as to limit distal displacement of the push rod beyond the distal position.

13. The device according to claim 1, wherein the push rod includes a push rod lumen defining an open proximal end and an open distal end, the push rod lumen configured for insertion of a guidewire therethrough.

14. The device according to claim 13, further comprising the guidewire disposed within the push rod lumen.

15. The device according to claim 5, wherein the skin nicking device includes an actuator operatively coupled with the push rod, the actuator configured to displace the push rod between the proximal position and the distal position.

16. The device according to claim 5, wherein the skin nicking device includes a locking mechanism configured to lock the push rod in at least one of proximal position or the distal position.

17. A catheter assembly, comprising:
a catheter including a catheter tube proximally coupled to a catheter hub having one or more extension legs proximally coupled therefrom, the catheter tube defining one or more lumens wherein each of the one or more lumens is in fluid communication with one of the extension legs; and
the catheter placement device according to any of the preceding claims, the catheter placement device coupled with the catheter.

18. The assembly according to claim 17, wherein the needle of the catheter placement device is inserted into one of the one or more lumens of the catheter tube.

19-26. (canceled)

27. A manufacturing method of a catheter placement device, comprising:
forming a slot extending through a needle wall of a needle;
placing a blade within the slot such that:
a sharp edge of the blade is directed radially outward with respect to the needle, and
the sharp edge is disposed inward of an outside surface of the needle; and
inserting a push rod within a needle lumen of the needle such that an angled surface of the push rod engages the blade.

28. The manufacturing method according to claim 27, further comprising hingedly coupling the blade to the needle wall such that the blade is pivotable through the slot.

29. The manufacturing method according to claim 27, further comprising forming a protrusion on an inside surface of the needle lumen to define a push rod barrier configured to limit distal displacement of the push rod within the needle lumen.
